(19) United States
(12) Patent Application Publication
(10) Pub. No.: US 2022/0162592 A1
(43) Pub. Date: May 26, 2022

(54) DUPLEX-SPECIFIC NUCLEASE DEPLETION FOR PURIFICATION OF NUCLEIC ACID SAMPLES

(71) Applicant: ZYMO RESEARCH CORPORATION, Irvine, CA (US)

(72) Inventors: Justin LIN, Irvine, CA (US); Casey CRUZ, Irvine, CA (US); Ken VITTAYARUKSKUL, Irvine, CA (US)

(21) Appl. No.: 17/602,392

(22) PCT Filed: Apr. 8, 2020
(86) PCT No.: PCT/US2020/027267

§ 371 Date: Oct. 8, 2021

Related U.S. Application Data
(60) Provisional application No. 62/830,936, filed on Apr. 8, 2019.
(60) Provisional application No. 62/884,403, filed on Aug. 8, 2019.

(51) Int. Cl. C12N 15/10; C12Q 1/6806; C12N 9/22; C12N 9/12

(52) U.S. Cl. CPC C12N 15/1065; C12Q 1/6806; C12N 9/1276; C12N 9/22; C12N 15/1096

(57) ABSTRACT

Methods and devices are provided for the removal of unwanted species from a sample using duplex-specific digestion.

This application claims the benefit of U.S. Provisional Patent Application Nos. 62/830,936, filed Apr. 8, 2019; and 62/884,403, filed Aug. 8, 2019, both of which are incorporated herein by reference in their entirety.

### BACKGROUND

### 1. Field of the Invention

The present invention relates generally to the field of molecular biology. More particularly, it concerns methods for ribosomal RNA depletion from samples for total RNA sequencing.

### 2. Description of Related Art

The RNA molecules present in cells are mostly rRNA species, whereas other coding and non-coding transcripts constitute only 1-15% of total RNA. Therefore, efficient enrichment of mRNA is a critical step for successful total RNA-seq experiments. A number of strategies exist for the removal of ribosomal RNA species and other high-abundance nucleic acid sequence species of low meaningful significance from either raw RNA sample material, or processed DNA libraries representing RNA transcripts for high-throughput sequencing analysis.

One class of methods rely on external probe hybridization of the raw RNA sample (probe-based methods), and depletion by either substrate-linked pull-down or enzymatic digestion of rRNA targeted by external probes. It has been shown that these methods have significant and measurable off-target effects on the profile of RNA species in the sample. A second class of methods rely on target denaturation and renaturation kinetics and a duplex-specific nuclease for abundant species depletion in previous duplex-specific nuclease (DSN) methods. This method has been demonstrated to deplete rRNA from bacterial total RNA samples, as commercial probe sets for non-mammalian rRNAs had not been available until recently. These methods are employed on adapterized DNA libraries, derived from processed RNA sample material. However, this approach is not as efficient as probe-based depletion, and there is an unmet need for improved methods of removing ribosomal RNA or other highly abundant RNA transcripts without significant off-target effects.

### SUMMARY

In a first embodiment, the present disclosure provides a method for the purification of nucleic acid samples comprising: (a) obtaining a nucleic acid sample; (b) performing reverse transcription on said sample and purifying to obtain a hybrid DNA/RNA library; and (c) depleting said DNA/RNA library of highly abundant, complementary DNA-RNA sequences using a duplex-specific nuclease (DSN), thereby obtaining a purified sample enriched for coding messenger RNA (mRNA) and non-coding transcripts (ncRNA) free of highly abundant repetitive sequences prior to preparation of a double-stranded DNA NGS library. In some aspects, the non-bacterial DNA/RNA libraries, are human, mouse, rat, and/or plant libraries. In some aspects, a method further comprises increasing the efficiency of depletion by performing DSN digestion on DNA-RNA hybrids at temperatures permissive of transient DNA-RNA hybrid interactions. In certain aspects, a method further comprises reducing the off-target bias of depletion by adding a denaturant to minimize mis-matched DNA-RNA sequence hybridization. In further aspects, a method further comprises purification of cDNA from the DSN depletion reaction for construction of NGS library from single-stranded cDNA to a dsDNA NGS library. In yet further aspects, a method further comprises comparison of depleted to undepleted samples using (e.g., peer-reviewed) statistical methods to assess off-target activity of rRNA depletion methods.

In some aspects, the nucleic acid sample is an RNA sample. In particular aspects, obtaining said RNA sample comprises extracting total RNA from a biological sample. In certain aspects, the biological sample is a human sample, such as saliva, tissue, or urine.

In certain aspects, reverse transcription comprises adding random hexamers and a reverse transcriptase to said sample. In specific aspects, said reverse transcriptase is MMLV reverse transcriptase.

In additional aspects, the method further comprises denaturing the DNA/RNA library prior to step (c). In some aspects, denaturing is performed at 80-90° C. In certain aspects, said sample is slowly cooled to minimize off-target annealing.

In some aspects, the method further comprising hybridizes the DNA and RNA to form DNA/RNA duplexes prior to step (c). In certain aspects, the DNA/RNA library sample is in a buffer with NaCl and denaturant.

In particular aspects, depleting is performed for 30-60 minutes, such as 35, 40, 45 50, 55, or 60 minutes. In some aspects, depleting is stopped by the addition of EDTA. In certain aspects, depleting comprises digestion of the DNA in the DNA/RNA duplexes.

In some aspects, the method removes unwanted abundant species from said sample. In certain aspects, the unwanted species comprises ribosomal RNA (rRNA). In some aspects, the purified sample comprises less than 10% rRNA, such as less than 5%, 4%, 3%, 2%, 1%, or 0.5% rRNA.

In particular aspects, the method results in a correlation coefficient of true abundance versus measured abundancies greater than 0.9, such as greater than 0.95, 0.96, 0.97, 0.98, or 0.99.

In additional aspects, the method further comprises generating a sequencing library from said the purified sample. In some aspects, DSN depletion is performed prior to preparing a sequencing library. In additional aspects, the method further comprises performing high-throughput sequencing on said sequencing library.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic depicting duplex-specific nuclease (DSN) use in abundant sequence reduction in previous methods (left) and the Present Methods (right).

FIG. 2: Schematic depicting DSN mechanism in previous methods (top) and the Present Methods (bottom).

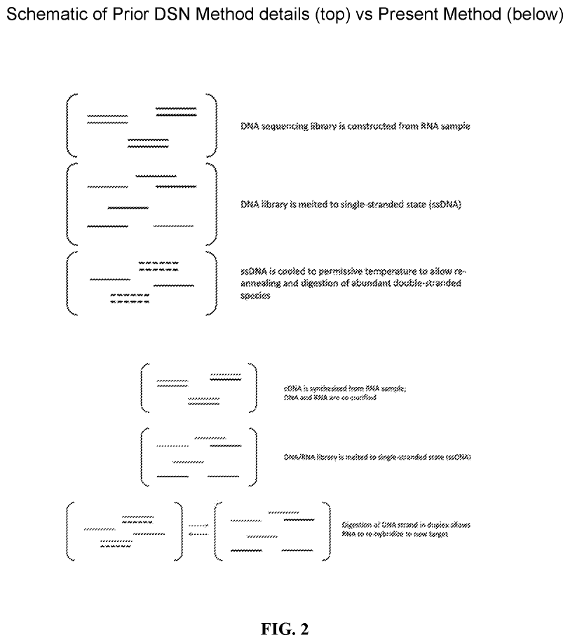

FIG. 3: Schematic depicting depletion of reads mapping to rRNA from NGS libraries in Prior DSN methods, prepared from E. coli samples (top, adapted from Yi et al.) and improved depletion of reads mapping to rRNA from NGS libraries in Present Methods (bottom).

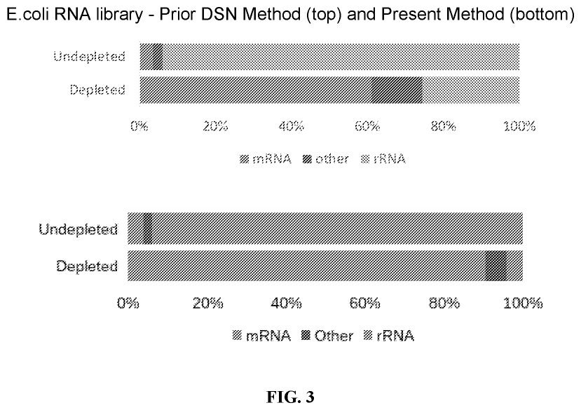

FIG. 4: Schematic depicting depletion of reads mapping to rRNA from NGS libraries in Present Methods, applied to both mammalian and bacterial samples, demonstrating the universality of the Present Methods.

FIG. 5: Comparison of rRNA depletion. Stacked bar plots representing depletion by the Present Methods and commercial Probe-based rRNA depletion methods using identical RNA-seq library preparation methods. Both depletion methods remove rRNA sequences efficiently.

FIG. 6: Comparison of off-target bias. Scatterplots comparing non-rRNA transcript correlation between Mock, Zymo (Present Methods), and Competitor R (Probe-based method) transcript abundances (Y axis), and Control transcript abundances (X axis) on real, non-rRNA genes. Perfect correlation between samples is 1.0. Mock (as expected) and Zymo depletion methods demonstrate near perfect correlation, while Competitor R demonstrates a lower correlation, with a significantly lower (up to 9%) coefficient of correlation.

FIG. 7: Quantification of off-target bias. MA plots (log ratio vs average intensity) of Depleted/Undepleted samples visualize the differences between treated and untreated RNA libraries using Zymo (Present Methods) and Competitor R (Probe-based method). The method “apeglm” is used as a Bayesian shrinkage estimator for effect size (Zhu et al), while the DESeq2 package is used as a statistical test for differential expression using a negative binomial generalized linear model (Love et al). Genes affected by treatment that pass multiple-test adjustment (p.adj<0.05) are highlighted in red, and are tallied above the plot. Zymo (Present Methods) depletion affects only 264 out of 20,004 mRNA genes, while Competitor R (Probe-based method) affects as much as 3854 genes out of 20,004.

FIG. 8: ERCC Spike-in measurement. Scatterplots of ERCC Spike-in control transcripts in RNA-seq libraries that have undergone Zymo treatment for rRNA depletion. High R-value indicates high correlation between true abundances of control transcripts (92 unique individual standards), and measured abundances in two separate Spike-in pools. Perfect correlation between any two samples would be 1.0. The high correlation coefficient of 0.95 demonstrates the high level of specificity, and minimal off-target effects.

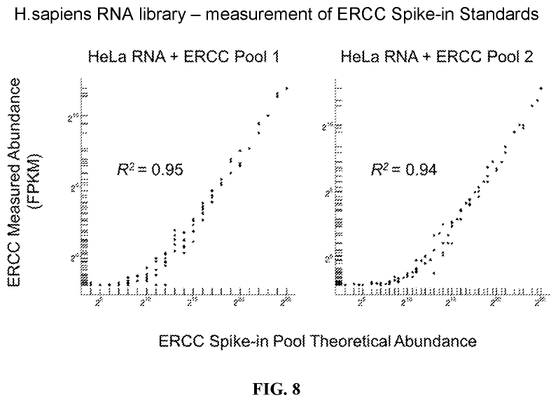

FIG. 9: Eliminating off-target depletion. Barplot from qPCR experiment demonstrating the elimination of off-target activity of the depletion treatment. Control Genes 1 and 2 represent mRNA transcripts not affected by depletion, while Abundant RNA and Off-target Gene represent RNA transcripts affected by depletion in Prior Methods. Bars represent normalized abundances of these RNAs in the sample. From left to right, “Input” represents the sample only “No Den.” represents the standard reaction conditions as found in previous methods. “+Den.”—in red, the embodiment reaction with the addition of a denaturant, such as those listed below. “Untreated” represents the reaction in the absence of incubation, while “Control Digest” represents the reaction in the presence of enzyme alone. The off-target depletion is mitigated in the presence of denaturant (e.g. non-ionic detergents such as saponins, N-dodecyl-beta-maltoside, and denaturants such as glycerol, ethylene glycol, 1,2-proanediol, DMSO, Urea, Guanidine-HCL, Betaine, and other similar compounds that reduce and/or inhibit secondary structure formation). It was found that the effective range of a denaturant such as DMSO to be between 5% to 10% v/v of the reaction and NaCl, 250 mM. However, a precise concentration would need to be titrated for each denaturant specifically, and for each sample type. Sample types compatible with this type of method would include, but are not limited to tissues, cell-free liquid, or cells from mammals, plants, insects, reptiles, bacteria, viruses, or synthetic nucleic acid samples.

### DESCRIPTION OF ILLUSTRATIVE EMBODIMENTS

Certain embodiments of the present disclosure provide methods for the purification of a biological sample for the removal of unwanted abundant species, such as ribosomal RNA. The method may comprise duplex-specific digestion on DNA-RNA hybrid duplexes. The method may further comprise adjusting the reaction buffer composition to improve the specificity of target depletion.

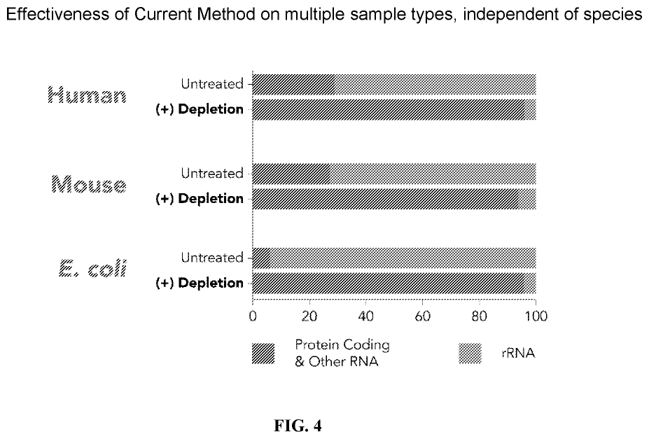

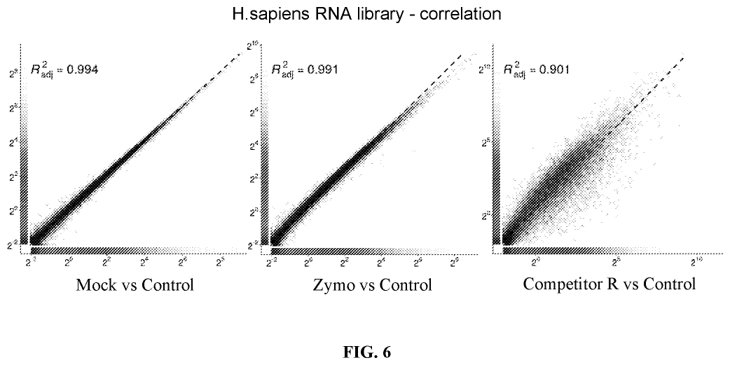

Specifically, the method may comprise reverse-transcription of cDNA from RNA using random hexamer priming and a reverse transcriptase, such as either MMLV or AMV reverse transcriptase, from which DNA and RNA are co-purified. Next, the DNA/RNA hybrid fragments may be denatured to a single-stranded state. The buffer may comprise a reagent to reduce off target bias such saponins, N-dodecyl-beta-maltoside, SDS, glycerol, ethylene glycol, 1,2-propanediol, DMSO, Urea, Guanidine-HCl, and/or Betaine, and a duplex-specific nuclease can be used to deplete the sample of complementary DNA-RNA fragments before next generation sequencing (NGS) double strand (ds) library preparation. Digestion of the DNA stand in the duplex allows for RNA to be re-hybridized to a new target, enabling multi-turnover kinetics. By contrast, previous methods employing DSN digestion of complementary DNA-DNA fragments of adapterized library fragments are limited to single-turnover reaction kinetics, as both strands are destroyed by digestion. In this new embodiment, the reaction is improved further, by adjusting the sample buffer composition to eliminate off-target depletion resulting from imperfect, off-target hybridization. In further improvements, the duplex is digested in a reagent and at a temperature that is permissive to transient hybridization, reducing further off-target digestion and increasing reaction kinetics. The DSN depleted sample is thus purified by removal of unwanted species, such as ribosomal RNA (rRNA) including 28s rRNA and 18S rRNA (see FIGS. 4 and 6). Specifically, the Present Method result in less than 3% rRNA, such as less than 2%, specifically about 1% rRNA (Table 1). The sample processed by the Present Methods has enriched levels of protein coding RNA and other transcripts of interest to the researcher.

The sample can then be processed for library preparation of double-stranded DNA which is then sequenced. The Present Methods take advantage of the higher rate of digestion kinetics due to the use of RNA as the complementary sequence in the duplex digestion.

In certain embodiments, the Present Methods do not comprise the steps of enriching for mRNA using mRNA-specific polyA tail selection or an oligo(dT) primer approach. In specific aspects, the DSN depletion is performed prior to sequencing library preparation in contrast to previous methods which comprise preparing a sequencing library and then performing DSN normalization.

### I. Purification of Nucleic Acid Samples

A. Sample Processing

The starting total RNA sample for the Present Methods can be obtained from any biological sample, such as soil, microbial fermentation, water, biofilms, and/or eukaryotic cellular cultures or biological body fluids (e.g. sputum, feces, lymph fluid, cerebrospinal fluid (CSF), urine, serum, sweat, various aspirates, and other liquid biological sources) and solid tissues.

The samples may be obtained from a variety of different sources, depending on the particular application being performed, where such sources include organisms that comprise nucleic acids, i.e. viruses; prokaryotes, e.g. bacteria, archaea and cyanobacteria; and eukaryotes, e.g. members of the kingdom protista, such as flagellates, amoebas and their relatives, amoeboid parasites, ciliates and the like; members of the kingdom fungi, such as slime molds, acellular slime molds, cellular slime molds, water molds, true molds, conjugating fungi, sac fungi, club fungi, imperfect fungi and the like; plants, such as algae, mosses, liverworts, hornworts, club mosses, horsetails, ferns, gymnosperms and flowering plants, both monocots and dicots; and animals, including sponges, members of the phylum cnidaria, e.g. jelly fish, corals and the like, combjellies, worms, rotifers, roundworms, annelids, molluscs, arthropods, echinoderms, acorn worms, and vertebrates, including reptiles, fishes, birds, snakes, and mammals, e.g. rodents, primates, including humans, and the like. Particular samples of interest include biological fluids, e.g., blood, plasma, tears, saliva, urine, tissue samples or portions thereof, cells (including cell linear, cell lines, cell cultures etc) or lysates thereof, etc. The sample may be used directly from its naturally occurring source and/or preprocessed in a number of different ways, as is known in the art.

The biological sample may be subjected to lysis to isolate nucleic acids for analysis. In particular embodiments, the sample is contacted with a lysis buffer (e.g., containing buffering agents, chaotropic salts, ionic detergents, non-ionic detergents solvents, EDTA, Trizol, monovalent and divalent salts). In some embodiments, the present disclosure provides appropriate salts (e.g. NaCl, KOH, MgCl₂, etc.) and salt concentration (e.g. high salt, low salt, 1 mM, 2 mM, 5 mM, 10 mM, 20 mM, 50 mM, 100 mM, 200 mM, 500 mM, 1 M, 2M, 3M, 4M, 5M, etc.) for use with the array of sample containers (e.g., a plurality of beads). In some embodiments, buffers for use with the array of sample containers (e.g., a plurality of beads) may include, but are not limited to H₃PO₄/NaH₂PO₄, Glycine, Citric acid, Acetic acid, Citric acid, MES, Cacodylic acid, H₂CO₃/NaHCO₃, Citric acid, Bis-Tris, ADA, Bis-Tris Propane, PIPES, ACES, Imidazole, BES, MOPS, NaH₂PO₄/Na₂HPO₄, TES, HEPES, HEPPSO, Triethanolamine, Tricine, Tris, Glycine amide, Bicine, Glycylglycine, TAPS, Boric acid (H₃BO₃/Na₂B₄O₇), CHES, Glycine, NaHCO₃/Na₂CO₃, CAPS, Piperidine, Na₂HPO₄/Na₃PO₄, and combinations thereof.

As indicated above, total RNA can be isolated from one or more cells, bodily fluids or tissues. An array of methods can be used to isolate total RNA from samples such as swabs, blood, sweat, tears, lymph, urine, saliva, semen, cerebrospinal fluid, amniotic fluid, feces, soil, water, sludge, etc. DNA can also be obtained from one or more cell or tissue in primary culture, in a propagated cell line, a fixed archival sample, forensic sample or archeological sample. Yeast species (e.g. Saccharomyces cerevisiae), fungi species, other microorganisms, human (Homo sapiens) liquid tissue (e.g. sputum, lymph fluid, cerebrospinal fluid (CSF), urine, serum, sweat, various aspirates, and other liquid biological sources) solid tissue, or tissue from a variety of species commonly used in diagnostic, research or clinical laboratories are contemplated as compatible with this purification procedure as sources of DNA and are all alternative embodiments of the present disclosure.

In certain embodiments, the Present Methods further comprise the purification and analysis of the DNA and/or RNA released from the sample using sheer or compression or tensile forces. The further analysis may comprise, for example, RNA gene sequencing.

Isolation of DNA and RNA is well known in the art. In particular embodiments, DNA isolation is performed using a commercially available kit such as the ZymoBIOMICS™ DNA Mini Kit. In particular aspects, the isolation is performed free of PCR inhibitors, such as polyphenols, humic and fulvic acids). In exemplary methods, plasmid isolation comprises modified mild alkaline lysis of host cells containing a plasmid, sodium hydroxide (NaOH) and sodium dodecyl sulphate (SDS), NaOH/SDS, denaturation, and precipitation of unwanted cellular macromolecular components as an insoluble precipitate, coupled to column-based silica, or other chromatography or purification methods. Isolation buffers based on alkaline lysis protocols are well known in the art and variations of compositions are contemplated as embodiments of the present invention that are compatible with various commercially available chromatographic columns and technologies. Alkaline lysis procedures generally use sodium acetate, potassium acetate, as well as a variety of other salts, including chaotropic salts. Ribonuclease RNAase A is commonly added to degrade contaminating RNA from the lysate. The clarification of the lysate can be performed by centrifugation or filtration methods both of which are known in the art. The plasmid is pure, typically with an OD260/280 ratio above 1.8. The plasmid DNA is suitably pure for use in the most sensitive experiments.

A number of methods have been used to isolate DNA from samples. For example, U.S. Pat. No. 5,650,506 relates to modified glass fiber membranes which exhibit sufficient hydrophilicity and electropositivity to bind DNA from a suspension containing DNA and permit elution of the DNA from the membrane. The modified glass fiber membranes are useful for purification of DNA from other cellular components. U.S. Pat. Nos. 5,705,628 and 5,898,071 disclose a method for separating polynucleotides, such as DNA, RNA and PNA, from a solution containing polynucleotides by reversibly and non-specifically binding the polynucleotides to a solid surface, such as a magnetic microparticle. A similar approach has been used in a product, “DYNABEADS DNA Direct” marketed by DYNAL A/S, Norway. Similarly, glass, plastic and other types of beads have been used to bind to and isolate DNA from solutions. Commercially, ZymoResearch offers the ZymoBIOMICS™-96 MagBead DNA Kit which includes beads for homogenization of diverse samples.

In some aspects, the nucleic acid is isolated as described by Ruggiere et al. (Springer Protocols Handbooks, Sample Preparation Techniques for Soil, Plant, and Animal Samples, 41-52, 2016; incorporated herein by reference). For example, phase separation techniques utilizing phenol-chloroform or acid guanidinium thiocyanate-phenol-chloroform extraction (e.g., Tri-Reagent® or Trizol® by commercial suppliers MRC and Invitrogen, respectively) and column-based separation techniques (that use a solid phase carrier such as silica or anion exchange resins) are the most prevalent methods used for nucleic acid isolation. Other technologies have also been employed for the binding and purification of nucleic acid including nitrocellulose, polyamide membranes, glass particles (powder or beads), diatomaceous earth, and anion-exchange materials (such as diethylaminoethyl cellulose).

Organic phase extraction of nucleic acids involves adding phenol and chloroform to a sample. The result is the formation of a biphasic emulsion which, upon centrifugation, the organic-hydrophobic solvents containing lipids, proteins, and other cellular components will settle on the bottom of the aqueous layer that contains the nucleic acids (Kirby, 1956; Grassman & Deffner, 1953; Tan & Yiap, 2009). The aqueous phase is subsequently partitioned from the organic layer for use in the precipitation of the nucleic acids. Ethanol (or isopropanol) with ammonium acetate (or some ionic salt) is used to precipitate the nucleic acids from the partitioned aqueous layer (Tan & Yiap, 2009). The nucleic acid is pelleted by centrifugation, washed with ethanol, and then resuspended in the desired low-salt solution (usually water or TE) for use in downstream analysis.

Due to the inherent nature of the chemistry of organic separation, DNA and RNA can be co-purified or selectively isolated individually. To selectively isolate DNA, an RNase A treatment may be necessary to remove RNA present in the aqueous layer (Rogers and Bendich, 1985). For effective DNA isolation, the aqueous layer must have a basic pH. Acidification using acid guanidinium thiocyanate-phenol-chloroform extraction, forces DNA to be partitioned into the interphase and organic phase, allowing for convenient isolation of RNA directly from the aqueous phase (Chomczynski & Sacchi, 1987 and Chomczynski et al., 1989).

In column-based separation, such as silica-based methods, use of a chaotropic agent, such as guanidinium chloride, will cause nucleic acids to selectively (and reversibly) bind to silica particles. The silica-nucleic acid-bound complexes can be subsequently washed with an alcohol solution to remove contaminants and then the nucleic acids eluted using water or TE. Spin-column extractions are well characterized and highly consistent due to reduced handling compared to phenol-chloroform extractions (Price et. al., 2009). They allow for quick and efficient purification by circumventing many of the problems associated with organic-phase separation such as incomplete phase separation and hassle of working with highly toxic solvents (Tan & Yiap, 2009).

B. Total RNA Purification Method

Several methods are available for the purification of RNA, such as described above. For example, the Zymo Quick-RNA™ MiniPrep Plus kit may be used to purify high-quality total RNA. In addition, Zymo DNA/RNA Shield™ ensures nucleic acid stability during sample storage/transport at ambient temperatures. In one exemplary method, RNA may be purified by the methods described in U.S. Pat. No. 9,051,563, incorporated herein by reference. In general, the method comprises (a) obtaining sample comprising a nucleic acid molecule and phenol and (b) contacting the sample to a silica substrate in the presence of a binding agent comprising a chaotropic salt, an alcohol or a combination thereof, thereby binding the nucleic acid molecule to the silica substrate. In certain aspects, a nucleic acid containing sample may comprise a substantial amount of phenol, such as about or greater than about 10%, 20%, 30%, 40% or 50% phenol by volume. A binding agent may comprise an alcohol such as a lower alcohol, e.g., methanol, ethanol, isopropanol, butanol or a combination thereof.

The addition of a chaotropic salt may be used for cell lysis and the formation of an RNA-containing precipitate. The term chaotropic salt refers to a substance capable of altering the secondary or tertiary structure of a protein or nucleic acid, but not altering the primary structure of the protein or nucleic acid. Examples of chaotropic salts include, but are not limited to, guanidine thiocyanate, guanidine hydrochloride sodium iodide, potassium iodide, sodium isothiocyanate, and urea. Guanidine salts other than guanidine thiocyanate and guanidine hydrochloride may be used as a chaotropic salts in the subject methods. Preferred chaotropic salts for use in the Present Methods are guanidine hydrochloride and guanidine thiocyanate. The concentration of chaotropic salt used to elicit RNA-containing precipitant formation may vary in accordance with the specific chaotropic salt selected. Factors such as the solubility of the specific salt must be taken into account. Routine experimentation may be used in order to determine suitable concentration of chaotropic salt for eliciting RNA-containing precipitate formation. In embodiments of the Present Methods employing guanidine hydrochloride as the chaotropic salt, the concentration of guanidine hydrochloride in the nucleic acid containing solution from which the RNA-containing precipitate is obtained is in the range of 1 M to 3 M, 2 M being particularly preferred. In embodiments of the Present Methods employing guanidine thiocyanate as the chaotropic salt, the concentration of guanidine thiocyanate in the nucleic acid-containing solution from which the RNA-containing precipitate is obtained is in the range of 0.5 M to 2 M, 1 M being particularly preferred. Combinations of chaotropic salts may be used to elicit RNA-containing precipitate formation. In embodiments of the invention employing multiple chaotropic salts, the chaotropic salts may be added in the form of concentrated solution or as a solid (and dissolved in the initial RNA-containing preparation).

After the addition of the chaotropic salts, the solution is allowed to incubate for a period of time sufficient to permit an RNA-containing precipitate to form. Unless the incubation conditions are modified during incubation, e.g., a temperature change, the longer the period of incubation time, the larger the quantity of RNA precipitate that will form. Incubation preferably occurs under constant temperature conditions. When a sufficient quantity of RNA precipitate for the purpose of interest, e.g., cDNA library formation, is formed, the RNA precipitate may be collected. The quantity of RNA precipitate formed may be monitored during incubation. Monitoring may be achieved by many methods, such methods include visually observing the formation of the precipitate (e.g., visually), collecting the precipitate during the incubation process and the like. In most embodiments of the invention, incubation time is at least one hour, preferably incubation is at least eight hours. Periods for incubation may be considerably longer than eight hours; no upper limit for incubation time is contemplated although need to obtain isolated RNA in a reasonable amount of time may be a constraint.

The temperature of the mixture formed by adding the chaotropic salt to the RNA-containing composition of interest, e.g., mixed microbial sample, influences the amount of RNA-containing precipitate formed in the subject method. In general, a greater precipitate yield will be obtained at a lower temperature, i.e., below room temperature. Preferably, freezing is avoided; however, a RNA-containing precipitate may form if a fresh cellular lysate is rapidly frozen. Additionally, lower temperatures may be used to reduce the activity of RNAses or detrimental chemical reactions occurring in the processed sample. Preferably, the temperature of the solution from which the RNA-containing precipitate formed is in the range of 1° C. to 25° C., more preferably in the range of 4° C. to 10° C.

After the RNA-containing precipitate has formed, the RNA-containing precipitate is collected. Collection entails the removal of the RNA-containing precipitate from the solution from which the precipitate was formed. The precipitate may be separated from the solution by any of the well-known methods for separation of a solid phase from a liquid phase. For example, the RNA-containing precipitate may be recovered by filtration or centrifugation. Many types of filtration and centrifugation systems may be used to collect the RNA-containing precipitate. Precautions against RNA degradation should be taken during the RNA precipitate collection step, e.g., the use of RNAase-free filters and tubes, reduced temperatures.

After the RNA-containing precipitate has been recovered, the precipitate may optionally be washed so as to remove remaining contaminants. A variety of wash solutions may be used. Wash solutions and washing conditions should be designed so as to minimize RNA losses from the RNA-containing precipitate. Preferably a wash solution containing the same chaotropic salt used to form the RNA-containing precipitate is used to wash the collected RNA-containing precipitate. The concentration of the chaotropic salt in the wash solution is preferably high enough for an RNA-containing precipitate to form, thereby minimizing losses of the RNA-containing precipitate during the washing process. Additionally, the washing solution is preferably at a temperature sufficiently low for RNA-containing precipitates to form, thereby minimizing losses of the RNA-containing precipitate during the washing process.

The collected RNA-containing precipitate may be solubilized so as to enable subsequent manipulation of the purified RNA in solutions. Solubilization may be accomplished by contacting the collected RNA-containing precipitate with a solution that does not elicit the formation of an RNA-containing precipitate. Typically, such a solution is an aqueous buffer (low ionic strength) or water. Examples of such buffers includes 10 mM Tris-HCl (pH 7.0), 0.1 mM EDTA; suitable buffering agents include, but are not limited to, tris, phosphate, acetate, citrate, glycine, pyrophosphate, aminomethyl propanol, and the like. The RNA-containing precipitate and the solution may be actively mixed, e.g., by vortexing, in order to expedite the solubilization process.

C. DSN Depletion

In certain embodiments, the present disclosure concerns cDNA preparation from the total RNA sample to prepare a cDNA/RNA hybrid fragments using random hexamers and a reverse transcriptase for use in making a NGS ds library. The reverse transcriptase may be selected from the group consisting of MMLV, ASLV, RSV, AMV, RAV, MAV, and HIV reverse transcriptases. In specific aspects, the reverse transcriptase is a MMLV reverse transcriptase.

The cDNA/RNA sample may be adjusted to an concentration of NaCl and denaturant, fully denatured at near boiling temperatures, and slowly cooled to a temperature minimizing off-target annealing. In some aspects, the sample is adjusted to have a NaCl concentration of up to about 1.0 M. For example, the concentration can be 10 mM, 200 mM, 250 mM, 300 mM, 350 mM, 400 mM, 450 mM, 500 mM, 600 mM, 700 mM or 800 mM to about 1.0M. In further aspects, the sample is adjusted have a DMSO concentration of 0-20%, such as 5%-20%, 5%-15%, 5%-10% or 8% to 12%. In certain embodiments, buffer composition may comprise the addition of a detergent or denaturant such as saponins, N-dodecyl-beta-maltoside, SDS, glycerol, ethylene glycol, 1,2-propanediol, DMSO, Urea, Guanidine-HCl, Betaine, etc. to improve the specificity of DNA/RNA hybridization, and further reduce off-target annealing. DSN enzyme may be added to this reaction mixture, and incubated for up to 1 hr, before quenching with EDTA.

In certain embodiments, the duplex-specific nuclease is selected from the group consisting of a Kamchatka Crab DSN, Gammarus putative nuclease, Glass shrimp putative nuclease, Mangrove fiddler crab putative nuclease, Kamchatka crab DNase K, a DNase I nuclease, and sea urchin Ca2+-Mg2+-dependent endonuclease.

D. Next Generation Sequencing

After purification, the DNA can be processed based on methods known in the art for the specific sequencing platform. Common next-generation sequencing platforms cover 100-600 base pairs per single read with varying degrees of accuracy.

The amplified PCR products can then be sequencing using a next-generating sequencing platform, such as Illumina MiSeq, Roche 454, or Ion Torrent. Any high-throughput technique for sequencing can be used in the practice of the invention. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. Patent Publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

Of particular interest is sequencing on the Illumina® MiSeq platform, which uses reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct Genomics 11(1):3-11; herein incorporated by reference).

E. Methods of Use

In certain embodiments, the Present Methods concern the detection and characterization of nucleic acid sequences. In particular, the subject methods find use in applications where one wishes to selectively manipulate, e.g., process, detect, eliminate etc., DNA containing duplexes in the presence of one or more other types of nucleic acids, i.e., in a complex nucleic acid mixture.

Thus, in certain aspects, the Present Methods concern identifying a nucleic acid analyte in a sample (e.g., methods of identifying bacterial and viral strain nucleic acid analytes and species specific nucleic acid analytes in a sample; methods of expression analysis, methods of the detection of the specific PCR product(s), etc.); methods of detection of nucleic acid variants including single nucleotide polymorphisms (SNPs); and methods of nucleic acid sequencing.

### II. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Purification of RNA Samples

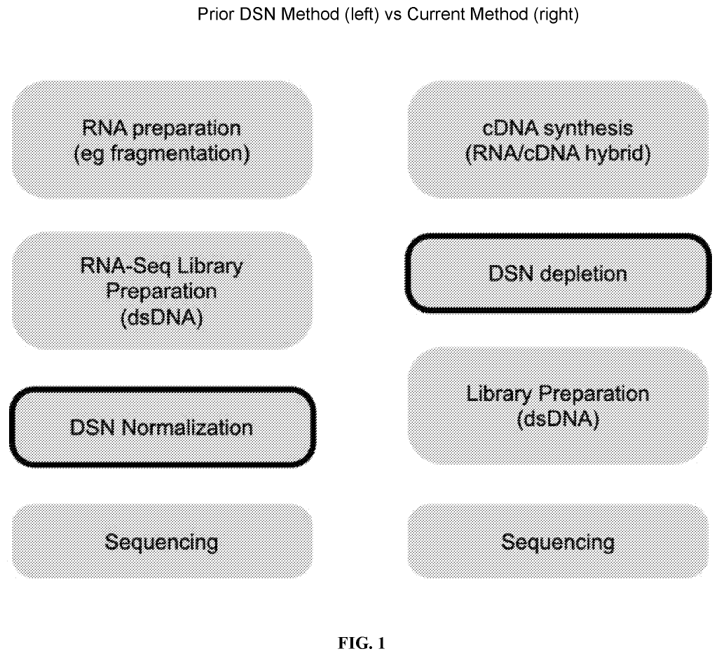

A method was developed and optimized for the removal of unwanted species from a starting RNA sample. FIG. 1 depicts the method for depletion of unwanted species using duplex-specific nuclease depletion

cDNA preparation: 500 ng of RNA with 0.5 ng ERCC RNA Spike-in standards was reverse transcribed to prepare a cDNA/RNA library using random hexamers and MMLV reverse transcriptase, and prepared using standard RT protocols.

DSN depletion: The above reaction containing the cDNA/RNA sample was adjusted to an optimized concentration of NaCl and denaturant to minimize off-target annealing, fully denatured at near boiling temperatures, and slowly cooled to a temperature permissive for transient hybridization, enabling multi-turnover reaction kinetics for greatly improved efficiency. DSN enzyme was added to this reaction mixture, and incubated for up to 1 hr, before quenching with EDTA.

qPCR analysis: cDNA was isolated from the above DSN depletion reaction using a purification column, and subjected to real-time PCR analysis using a SYBR-green dye and gene-specific primers.

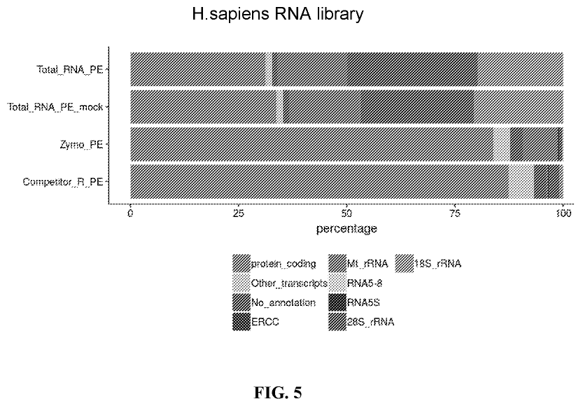

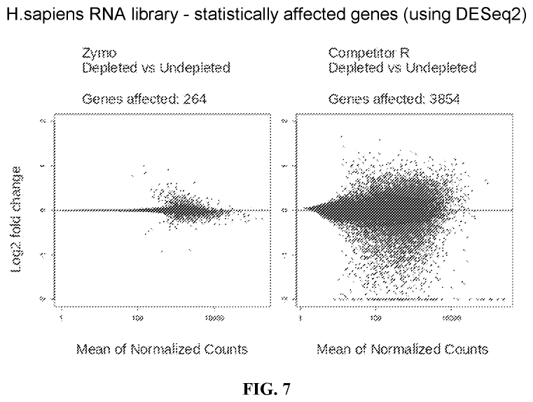

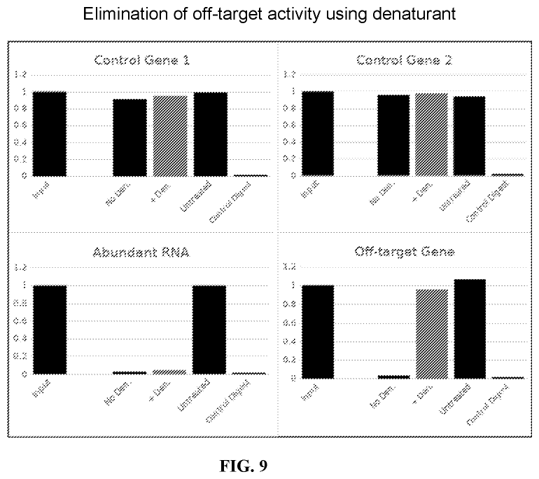

It was found that the samples subjected to the Present Methods of DNS depletion comprised a lower proportion of rRNA and other unwanted species as compared to previous methods (FIGS. 4-6). In addition, FIG. 7 shows the low off-target bias of the Present Methods as measured by the non-rRNA transcript correlation. The DSN depletion was also optimized to prevent off-target depletion (FIG. 9).

RNA-seq library preparation: cDNA from the DSN depletion reaction was converted to short-read sequencing libraries using a custom protocol. Briefly, oligonucleotides containing partial sequencing adapter sequence were ligated to the 3′ of the cDNA. Oligonucleotides complementary to the sequencing adapter were then used to synthesize the cDNA second strand. Double-stranded partial sequencing adapter was then ligated to the 5′ end of the DNA. Finally, barcode indexes were added to the sequencing library using standard PCR.

High-throughput sequencing: Sequencing was performed on the Illumina HiSeq, with an average of 50 million reads per sample. Sequencing images were then converted to fastq file format using the on-board sequencer software, while fastq trimming and read alignment were performed using in-house bioinformatics pipelines. Reads were then classified by Ensembl gene biotypes to create stacked bar plots of read categories, while read count correlation were compared between treated and untreated samples using a scatterplot and lm fit functions in R. Absolute abundances of ERCC transcripts were compared to read abundances in depleted RNA using a custom analysis workflow.

TABLE 1
Comparison of rRNA depletion. Percentages of reads mapping to various RNA classes, between Present
Methods and commercial Probe-based methods. Gene biotype table of read abundance from H. sapiens RNA libraries.

| Sample | Protein coding | Other | No annotation | Mitochondrial | 5.8S rRNA | 5S rRNA | 18S rRNA | 28S rRNA |
|---|---|---|---|---|---|---|---|---|
| Untreated | 31.24% | 1.57% | 1.17% | 16.23% | 0.01% | 0.05% | 30.01% | 19.72% |
| Mock | 33.67% | 1.65% | 1.23% | 16.57% | 0.01% | 0.05% | 26.21% | 20.61% |
| Zymo | 83.91% | 3.96% | 2.86% | 8.14% | 0.00% | 0.14% | 0.81% | 0.19% |
| Compeitor_R | 87.38% | 5.92% | 2.77% | 0.37% | 0.00% | 0.19% | 2.61% | 0.75% |

* * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Bogdanova E A, Barsova E V, Shagina I A, Scheglov A, Anisimova V, Vagner L L, Lukyanov S A, Shagin D A. Normalization of full-length-enriched cDNA. Methods Mol Biol. 2011a; 729:85-98. doi: 10.1007/978-1-61779-065-2_6/pmid: 21365485

Bogdanova E A, Shagina I A, Mudrik E, Ivanov I, Amon P, Vagner L L, Lukyanov S A, Shagin D A. DSN depletion is a simple method to remove selected transcripts from cDNA populations. Mol Biotechnol. 2009; 41 ((3)):247-53. doi: 10.1007/s12033-008-9131-y/pmid: 19127453

Bogdanova E A, Shagina I A, Yanushevich Y G, Vagner L L, Lukyanov S A, Shagin D A. Preparation of prokaryotic cDNA for full-scale transcriptome analysis. Russian Journal of Bioorganic Chemistry. 2011b; 37 (6):775-8. ISI:000297344600012 http://www.springerlink.com

Christodoulou D C, Gorham J M, Herman D S, Seidman JG. Construction of normalized RNA-seq libraries for next-generation sequencing using the crab duplex-specific nuclease. Curr Protoc Mol Biol. 2011; Chapter 4:Unit4.12. doi: 10.1002/0471142727.mb0412s94/pmid: 21472699

Kunitz M. Crystalline desoxyribonuclease; digestion of thymus nucleic acid; the kinetics of the reaction. J Gen Physiol. 1950; 33:363-377./pmid: 15406374

Liu M, Yuan M, Lou X, Mao H, Zheng D, Zou R, Zou N, Tang X, Zhao J. Label-free optical detection of single-base mismatches by the combination of nuclease and gold nanoparticles. Biosens Bioelectron. 2011; 26 (11):4294-300. doi: 10.1016/j.bios.2011.04.014/pmid: 21605966

Peng R H, Xiong A S, Xue Y, Li X, Liu J G, Cai B, Yao Q H. Kamchatka crab duplex-specific nuclease-mediated transcriptome subtraction method for identifying long cDNAs of differentially expressed genes. Anal Biochem. 2008; 372 (2):148-55./pmid: 17905189

Shagin D A, Rebrikov D V, Kozhemyako V B, Altshuler I M, Shcheglov A S, Zhulidov P A, Bogdanova E A, Staroverov D B, Rasskazov V A, Lukyanov S. A novel method for SNP detection using a new duplex-specific nuclease from crab hepatopancreas. Genome Res. 2002; 12 (12):1935-42./pmid: 12466298

Shagina I, Bogdanova E, Mamedov I Z, Lebedev Y, Lukyanov S, Shagin D. Normalization of genomic DNA using duplex-specific nuclease. Biotechniques. 2010; 48 (6):455-9. doi: 10.2144/000113422/pmid: 20569220

Swennenhuis J F, Foulk B, Coumans F A, Terstappen L W. Construction of repeat-free fluorescence in situ hybridization probes. Nucleic Acids Res. 2012; 40 (3):e20. doi: 10.1093/nar/gkr1123/pmid: 22123742

Yi H, Cho Y J, Won S, Lee J E, Jin Yu H, Kim S, Schroth G P, Luo S, Chun J. Duplex-specific nuclease efficiently removes rRNA for prokaryotic RNA-seq. Nucleic Acids Res. 2011; 39 (20):e140. doi: 10.1093/nar/gkr617/pmid: 21880599

Yin B C, Liu Y Q, Ye B C. One-step, multiplexed fluorescence detection of microRNAs based on duplex-specific nuclease signal amplification. J Am Chem Soc. 2012; 134 (11):5064-7. doi: 10.1021/ja300721s/pmid: 22394262

Zhao Y, Hoshiyama H, Shay J W, Wright W E. Quantitative telomeric overhang determination using a double-strand specific nuclease. Nucleic Acids Res. 2008; 36 (3):e14./pmid: 18073199

Zhao Y, Shay J W, Wright W E. Telomere G-overhang length measurement method 1: the DSN method. Methods Mol Biol. 2011; 735:47-54. doi: 10.1007/978-1-61779-092-8_5/pmid: 21461810

Zhulidov P A, Bogdanova E A, Shcheglov A S, Vagner L L, Khaspekov G L, Kozhemyako V B, Matz M V, Meleshkevitch E, Moroz L L, Lukyanov S A, Shagin D A. Simple cDNA normalization using kamchatka crab duplex-specific nuclease. Nucleic Acids Res. 2004; 32 (3):e37./pmid: 14973331

Zhu A, Ibrahim J G, Love M I (2018). “Heavy-tailed prior distributions for sequence count data: removing the noise and preserving large differences.” Bioinformatics. doi: 10.1093/bioinformatics/bty895.

What is claimed is:

1. A method for the purification of nucleic acid samples comprising:
(a) obtaining a nucleic acid sample;
(b) performing reverse transcription on said sample and purifying to obtain a hybrid DNA/RNA library;
(c) depleting said DNA/RNA library of highly abundant, complementary DNA-RNA sequences using a duplex-specific nuclease (DSN), thereby obtaining a purified sample enriched for coding messenger RNA (mRNA) and non-coding transcripts (ncRNA) free of highly abundant repetitive sequences prior to preparation of a double-stranded DNA NGS library.

2. The method of claim 1, further comprising increasing the efficiency of depletion by performing DSN digestion on DNA-RNA hybrids at temperatures permissive of transient DNA-RNA hybrid interactions;

3. The method of claim 1, further comprising reducing the off-target bias of depletion by adding a denaturant to minimize mis-matched DNA-RNA sequence hybridization.

4. The method of claim 1, further comprising purification of cDNA from the DSN depletion reaction for construction of NGS library from single-stranded cDNA to a dsDNA NGS library.

5. The method of claim 1, further comprising comparison of depleted to undepleted samples using statistical methods to assess off-target activity of rRNA depletion methods.

6. The method of claim 1, wherein the nucleic acid sample is an RNA sample.

7. The method of claim 6, wherein obtaining said RNA sample comprises extracting total RNA from a biological sample.

8. The method of claim 7, wherein the biological sample is a human sample.

9. The method of claim 8, wherein the sample comprises saliva, tissue, or urine.

10. The method of claim 1, wherein reverse transcription comprises adding random hexamers and a reverse transcriptase to said sample.

11. The method of claim 10, wherein said reverse transcriptase is MMLV reverse transcriptase.

12. The method of claim 1, further comprising denaturing the DNA/RNA library prior to step (c).

13. The method of claim 12, wherein denaturing is performed at 80-90° C.

14. The method of claim 13, wherein said sample is slowly cooled to minimize off-target annealing.

15. The method of claim 14, further comprising hybridizing the DNA and RNA to form DNA/RNA duplexes prior to step (c).

16. The method of claim 1, wherein the DNA/RNA library is a human mouse, rat or plant library.

17. The method of claim 1, wherein depleting is performed for 30-60 minutes.

18. The method of claim 1, wherein depleting is stopped by the addition of EDTA.

19. The method of claim 1, wherein depleting comprises digestion of the DNA in the DNA/RNA duplexes.

20. The method of claim 1, wherein the method removes unwanted abundant species from said sample.

21. The method of claim 20, wherein the unwanted species comprises ribosomal RNA (rRNA).

22. The method of claim 21, wherein the purified sample comprises less than 10% rRNA.

23. The method of claim 21, wherein the purified sample comprises less than 5% rRNA.

24. The method of claim 1, wherein the method results in a correlation coefficient of true abundance versus measured abundancies greater than 0.9.

25. The method of claim 1, wherein the method results in a correlation coefficient of true abundance versus measured abundancies greater than 0.95.

26. The method of claim 25, further comprising generating a sequencing library from said the purified sample.

27. The method of claim 26, wherein DSN depletion is performed prior to preparing a sequencing library.

28. The method of claim 26, further comprising performing high-throughput sequencing on said sequencing library.
